(19) United States
(12) Patent Application Publication
(10) Pub. No.: US 2024/0189496 A1
(43) Pub. Date: Jun. 13, 2024

(54) SURGICAL DRAIN FOR COLLECTION AND PRESERVATION OF TUMOR-ASSOCIATED BIOMARKERS IN SURGICAL DRAIN FLUID

(71) Applicant: Washington University, St. Louis, MO (US)

(72) Inventors: Jose Zevallos, St. Louis, MO (US); Ricardo Ramirez, St. Louis, MO (US); Benjamin Wahle, St. Louis, MO (US); Aadel Chaudhuri, St. Louis, MO (US)

(21) Appl. No.: 18/282,933

(22) PCT Filed: Mar. 18, 2022
(86) PCT No.: PCT/US22/20889

§ 371 Date: Sep. 19, 2023

Related U.S. Application Data
(60) Provisional application No. 63/163,639, filed on Mar. 19, 2021.

(51) Int. Cl. A61M 1/00; A61B 90/00

(52) U.S. Cl. CPC A61M 1/73; A61B 90/39; A61M 1/79; A61M 2205/3303; A61M 2205/3324; A61M 2205/3331; A61M 2205/3368; A61M 2205/7545

(57) ABSTRACT

A surgical drainage system is disclosed that includes an assay device operatively coupled to a surgical drainage tube. The drainage tube is configured for implantation within a surgical bed of a subject to drain surgical drain fluid out of the surgical bed. The assay device is configured to detect at least one analyte within the drainage fluid. The at least one analyte is indicative of a surgical bed condition.

### CROSS-REFERENCE TO RELATED APPLICATIONS

This application claims priority to U.S. Provisional Application No. 63/163,639, filed Mar. 19, 2021, the disclosure of which is incorporated herein by reference.

### STATEMENT REGARDING FEDERALLY SPONSORED RESEARCH OR DEVELOPMENT

Not applicable.

### FIELD OF THE DISCLOSURE

The present disclosure generally relates to devices and methods for drainage of a surgical bed for monitoring the condition of a subject using physiological measurements and surgical drain fluid samples obtained using a surgical drain tube.

### BACKGROUND OF THE DISCLOSURE

Surgery often requires major tissue resection and reconstruction. The most common complications of surgery is the buildup of fluids and infection. For example, a common complication associated with tumor resection is fistula formation. A fistula occurs when there is a leak in the surgical repair, resulting in fluids leaking into surrounding tissues and subsequently through the skin. Early recognition of a fistula is critical to limiting associated wound complications.

Fistula formation is often associated with non-specific signs and symptoms, including fever, erythema or tenderness, and leukocytosis. In the later stages, it is also associated with increased surgical drain output. As a result, the diagnosis is often delayed and associated with worse complications.

A need exists for devices, systems, and methods of monitoring various parameters within a surgical bed during recovery to detect complications such as incipient fistula formation, infection, leakage from adjoining organs, and other complications.

### SUMMARY OF THE INVENTION

The invention provides surgical drainage system for use in obtaining diagnostic information from the site of a surgical intervention. In one aspect, a drainage system of the invention comprises a collection device that is operatively coupled to a drainage tube that is implantable in a surgical bed. The drainage tube acts as a conduit for movement of drain fluid from the surgical bed to the collection device. The contents of the drainage fluid then can be analyzed for the presence of one or more antigens indicative of a disease or condition. In certain embodiments, the drainage tube is a suction drain tube that facilitates flow of drainage fluid from a wound or surgical bed to a collection device.

In a preferred embodiment, the collection device is configured to detect an analyte or other indicia of a disease or condition. In this embodiment, drain fluid is collected in the detection device, which contains elements necessary for analyte detection. For example, the detection device may contain a lateral flow antigen test. In that case, analysis of the presences of an analyte or biomarker indicative of a disease or condition can be performed at the point of care or later in a laboratory setting. In other embodiments, the collection device contains an assay device for detection of one or more analytes. The assay device can be in the form of a replaceable cartridge that houses, for example, a lateral flow antigen test. The detected antigen may be, for example, a tumor neoantigen or other antigenic component indicative of cancer or precancer. In addition to lateral flow tests, the assay device may be configured to identify bacteria, virus, parasites, circulating tumor DNA (ctDNA) or RNA associated with expression of tumor DNA. Finally, a device of the invention is useful for analysis of the immune complement present in a surgical bed.

In addition to the detection of cancer biomarkers, the invention is useful for the detection of bacterial, viral and/or fungal proteins and/or nucleic acids. Essentially, the onboard assay device in a system of the invention can be configured to detect any analyte or biomarker associated with any disease or condition. Apparatus and methods of the invention are also useful for the detection and/or identification of infection in the surgical bed, fistula formation, chyle leak, bile leak, anastomotic leak or any combination of the foregoing.

In one embodiment, a surgical drain system of the invention comprises a proximal portion configured for implantation in a surgical bed and a distal portion configured to drain surgical drain fluid away from the surgical bed via an inner lumen that is defined within the proximal and distal portions of the surgical drain tube. In a preferred configuration, the inner lumen is coated with one or more preservatives to maintain the integrity of the analytes to be detected. The preservative may be an agent that preserves nucleic acid, proteins, or other analytes to be detected. In preferred embodiments, the preservative is selected from ethylene diamine tetra acetic acid (EDTA), Sodium dodecyl sulfate (SDS) or a combination thereof.

Further, the lumen may comprise a one-way valve that is configured to prevent backflow of the drain fluid. The drain tube itself may be a suction tube or may be configured in association with a suction device to facilitate flow of drain fluid away from the surgical bed. In addition the lumen may be connected to a sample port comprising a valve and connection fitting configured to seal the valve when in a closed position and to direct drain fluid flow out of the lumen when in an open position. The connection fitting may contain a resealable lock (e.g., a Luer lock) configured to reversibly couple the accessory reservoir to the connection fitting. In some embodiments, a device or system of the invention further comprises an accessory reservoir that is reversibly coupled to the connection fitting and that is configured to receive a sample of the drain fluid. The accessory reservoir can be removed and transported for analysis of the drain fluid or may be subjected to point-of-care analysis. The accessory reservoir may contain a preservative similar to the lumen as described above.

In some embodiments, a system of the invention is used to perform serial sampling of the drain fluid via a plurality of accessory reservoirs that are serially connected and disconnected from the connection fitting. The accessory reservoir may also contain a filter configured to filter the drainage fluid as it passes into the accessory reservoir. The filter may additionally serve as a capture mechanism for retaining analytes for subsequent detection.

Also in a preferred embodiment, a system of the invention contains a heat source operatively coupled to at least a portion of the drain tube and/or the accessory reservoir. The heat source is configured to maintain the drain fluid at an appropriate temperature to facilitate measurement and to prevent degradation of the analyte to be detected. The lumen of the drain tube may further comprise an insulating material. The insulating material may enclose at least a portion of the drain tube and/or the accessory reservoir.

In some embodiments, a system of the invention comprises a sensor that is capable of monitoring a physiological condition (e.g., temperature, pH, pressure, hydration, oxygenation and others) and/or a disease state. In some alternatives, the sensor is integrated into the system and in others it is removable attached to the system. The invention contemplates the use of one or more sensors that are ideally placed at or near the proximal end of the drain. The sensors can be configured to monitor any condition or state of the surgical bed.

The invention also contemplates methods of using a device as describe herein for the monitoring of a physiological state or condition or for the detection and/or identification of disease. Such methods include, but are not limited to, applying a surgical drain system of the invention to a surgical bed, extracting drain fluid from the surgical bed, and analyzing the drain fluid for indicia of a disease and/or condition. In preferred embodiments, the disease and/or condition is selected from a bacterial infection, a viral infection, sepsis, an immune disorder, cancer or precancer, or an infectious agent (e.g., HPV, EBV and others).

### DESCRIPTION OF THE DRAWINGS

FIG. 1 is a schematic diagram illustrating a surgical drain in accordance with one aspect of the disclosure.

FIG. 2 is a schematic diagram illustrating a surgical drain system in accordance with one aspect of the disclosure.

Those of skill in the art will understand that the drawings, described below, are for illustrative purposes only. The drawings are not intended to limit the scope of the present teachings in any way.

There are shown in the drawings arrangements that are presently discussed, it being understood, however, that the present embodiments are not limited to the precise arrangements and are instrumentalities shown. While multiple embodiments are disclosed, still other embodiments of the present disclosure will become apparent to those skilled in the art from the following detailed description, which shows and describes illustrative aspects of the disclosure. As will be realized, the invention is capable of modifications in various aspects, all without departing from the spirit and scope of the present disclosure. Accordingly, the drawings and detailed description are to be regarded as illustrative in nature and not restrictive.

### DETAILED DESCRIPTION OF THE INVENTION

In various aspects, the invention presents a surgical drain system for obtaining surgical drain fluid from a surgical bed. The drain system may be a modification of a traditional surgical suction drain. Systems of the invention are useful for the detection of indicia of a disease or condition and provide a low-cost option for early detection of disease or surgical complications including, but not limited to, a salivary fistula. Systems of the invention are capable of continuously monitoring drain fluid for an analyte or biomarker indicative of a condition or disease. In another embodiment, systems of the invention are useful to identify surgical complications in real time. For example, systems of the invention are useful for detection of amylase in drain fluid in head and neck surgery as indicative of the formation of a fistula within the surgical bed.

In various aspects, surgical drain system of the invention incorporates an assay device, such as a lateral flow antigen test. The lateral flow antigen test monitors surgical drain fluid for an analyte indicative of the condition of a surgical bed including, but not limited to, amylase, a protein found in saliva.

In a preferred configuration, an assay device is built into the drainage tube or fluid reservoir and can signal clinicians if an analyte, such as amylase, is present in the wound bed prior to the clinical manifestation of a fistula. This early recognition is a basis for preemptive action (limiting oral intake, antibiotics, etc) and increased surveillance to prevent worsening complications from a fistula.

Thus, in one aspect, disclosed surgical drain systems provide low cost, early detection of one of the most common complications in head and neck surgery, salivary fistula. The incorporation of a lateral flow antigen test into a surgical drain may be used for the detection of other proteins and analytes of interest associated with a variety of other conditions. In various additional aspects, the lateral flow antigen test may be used to detect other proteins or analytes associated with other conditions or complications. Non-limiting examples of additional proteins or analytes associated with other conditions or complications that are detected using the lateral flow antigen test of the disclosed surgical drain system include markers of bacterial infection, chyle leaks in the neck, bile leaks in the abdomen after gall bladder surgery, anastomotic leaks after small bowel repair or colorectal surgery (detection of pepsin or amylase) and any other suitable markers.

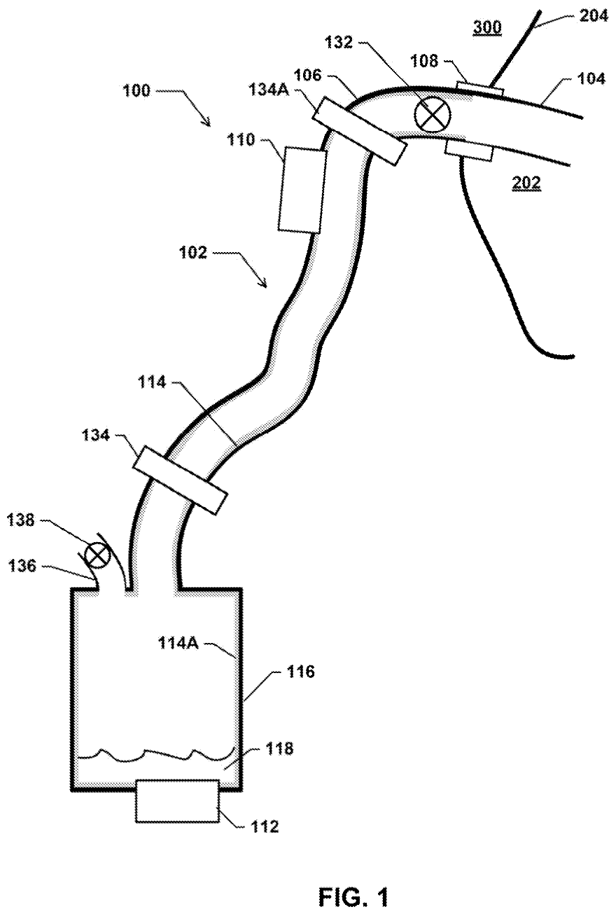

FIG. 1 is a schematic illustration of a surgical drain system 100 that includes the surgical drain tube 102 as described above. The surgical drain tube 102 includes a proximal portion 104 that is configured for implantation within a surgical bed 202 of a subject to drain surgical fluid out of the surgical bed 202 in various aspects. The surgical drain tube 102 further includes a distal portion 106 that projects into an external region 300 outside of the subject when the proximal portion 104 is implanted within the surgical bed 202. The surgical drain tube 102 further includes a skin interface 108 configured to form a sealed passage through the subject's skin 204 for the surgical drain tube 102 passing from the surgical bed 202 to the external region 300.

Referring again to FIG. 1, the distal portion 106 of the surgical drain tube 102 extends from the proximal portion 104 and skin interface 108 to a main reservoir 116 that collects the surgical drain fluid 118 from the surgical bed 202. In some aspects, the surgical drain fluid may passively flow from the surgical bed to the main reservoir. By way of non-limiting example, the flow of the surgical drain fluid may be drawn by gravity from the surgical bed to a main reservoir located below the surgical bed. In other aspects, the surgical drain fluid may be drawn by a vacuum pressure created and/or maintained within the main reservoir.

Referring again to FIG. 1, the surgical drain system 100 further includes a vacuum port 136 with a vacuum valve 138. The vacuum port is configured to remove air from the main reservoir 116, thereby creating and maintaining a vacuum pressure within the main reservoir 116. In various aspects, the vacuum pressure is sufficient to draw the surgical fluid 118 from the surgical bed 202 into the main reservoir 116 via the surgical drain tube 102. In some aspects, a vacuum source (not illustrated) may be operatively coupled to the vacuum port 136 to create and maintain a vacuum pressure within the main reservoir 116. In various aspects, the vacuum source may be any suitable medical vacuum source without limitation. Non-limiting examples of suitable vacuum sources include a suction line, a vacuum tank, a vacuum pump, and any other suitable vacuum source.

In various aspects, the vacuum valve 138 may be opened and closed using any suitable means including, but not limited to, manual opening and closing, actuated opening and closing, and any other suitable means of modulating the position of the valve 138. In some aspects, the vacuum valve 138 is opened to allow the vacuum source to create and/or maintain the vacuum pressure within the main reservoir 116 and closed to prevent repressurization within the main reservoir 116 if the vacuum source is inactivated or removed from the vacuum port 136. In other aspects, the position of the vacuum valve 138 may be modulated to one or more positions between fully opened and fully closed to modulate the level of vacuum pressure created and/or maintained within the main reservoir 116 by the vacuum source. In additional aspects, the operation of the vacuum source may be modulated as needed to control the level of the vacuum pressure within the main reservoir 116.

In some aspects, the vacuum pressure within the main reservoir 116 may be created manually as described below. In these aspects, at least a portion of the main reservoir 116 may be constructed using a flexible or elastic material that can be compressed or squeezed by a practitioner to displace an amount of air out of the main reservoir 116 through the opened vacuum port 136. The practitioner may then close the vacuum valve 138 of the vacuum port 136 and release the compressed main reservoir 116. Upon release, the elastic rebound of the flexible or elastic materials of the main reservoir 116 return the main reservoir 116 to its original uncompressed volume and create a vacuum pressure. In some aspects, the vacuum valve 138 may be a one-way valve that permits flow out of the main reservoir 116 and prevents backflow of air back into the main reservoir 116, obviating the need for manual opening and closing of the vacuum valve 138 during operation of the surgical drainage system 100.

In other additional aspects, the surgical drainage system 100 further includes a one-way backflow prevention valve 132 positioned within the distal portion 106 of the surgical drain tube 102 to permit flow from the surgical bed 202 to the main reservoir 116 and to prevent backflow toward the surgical bed 202. The backflow prevention valve 132 may be positioned at any suitable position between the skin interface 108 and the main reservoir 116 without limitation. In some aspects, the backflow prevention valve 132 is positioned relatively near the skin interface 108 to assure one-way flow through the distal portion 106 of the surgical drain tube 102. This one-way flow prevents the backflow of surgical drain fluid 118 back into the surgical bed 202, or the fouling of various additional elements of the surgical drainage system 100 described below including, but not limited to, filters and assay devices, due to undesired backflow within the surgical drain tube 102.

In some aspects, the one-way vacuum valve 138 and the backflow prevention valve 132 operate in a coordinated manner to facilitate the creation of vacuum pressure manually by compressing and releasing a flexible portion of the main reservoir 116 as described above. In these aspects, when the main reservoir 116 is compressed, the backflow prevention valve 132 prevents air from passing out of the proximal end 104 of the surgical drain tube 106, and the vacuum valve 138 releases air from the vacuum port 136 to create the vacuum pressure. Once the main reservoir 116 is released, air is prevented from re-entering the vacuum port 136 by the vacuum valve 138, and the backflow prevention valve 132 permits surgical drainage 118 to flow into the proximal end 104 of the surgical drain tube 106.

In some aspects, the surgical drain system provides for monitoring of the surgical drain fluids passing through the surgical drain fluid for analytes that are indicative of a complication associated with the surgical bed including, but not limited to, infections, fistula formations, inflammations, leaks from regions adjoining the surgical bed, rejection of transplanted cells, tissues, or organs, and any other surgical complication without limitation. Referring again to FIG. 1, the surgical drain system 100 further includes an assay device 110 operatively coupled to the surgical drain tube 102. In various aspects, the assay device 110 may be operatively coupled to the surgical drain tube 102 at any position along the distal portion 106 without limitation. In some aspects, the assay device 110 is operatively coupled to the distal portion 106 near the skin interface 108. In additional aspects, the assay device 110, or an additional assay device 112, is operatively coupled to the main reservoir 112. In some aspects, the assay device 110/112 may be provided in the form of a replaceable cartridge to provide for repeated monitoring of the drain fluids over an extended time period.

In various aspects, the assay device 110/112 may be any suitable type of assay device without limitation that is capable of detecting proteins and other analytes of interest associated with a variety of other conditions as described above. Non-limiting examples of suitable assay devise include immunoassays such as lateral flow immunochromatographic assay devices and any other suitable assay device without limitation.

Referring again to FIG. 1, the surgical drain system may further include additional features to condition the drain fluid as it passes through the drain tube 102. Referring again to FIG. 1, the surgical drain system 100 may further include at least one filter 134/134A operatively coupled to the distal portion 106 of the drain tube 102 in various aspects. The at least one filter 134/134A is configured to remove one or more components from the drain fluid 118 including, but not limited to, whole cells, clots, and any other component that may potentially interfere with the operation of the assay devices and/or subsequent processing and analysis of the drain fluid as described below. Any suitable medical-grade filter may be selected for use in the surgical drain system without limitation.

In various aspects, one or more elements of the surgical drain system may further include a coating over at least a portion of the surfaces contacting the drain fluid. In these aspects, the coating is provided to prevent or inhibit the degradation of proteins and nucleotides within the drainage fluid. The coating may include any one or more compounds that inhibit denaturing of proteins and/or nucleotides, coagulation, and any other type of degradation within the drainage fluid without limitation. Non-limiting examples of suitable coating compositions include EDTA, heparin, trisodium citrate (TSC) and any other suitable coating compound, and any combination thereof.

Referring again to FIG. 1, in some aspects, the surgical drain system 100 may further include an EDTA coating 114 over at least a portion of the inner surface of the distal portion 106 of the drainage tube 102. Without being limited to any particular theory, the coating is not provided to the proximal portion 104 of the drainage tube 102 because of the potential for an anticoagulant and/or anti-denaturing coating to disrupt healing within the surgical bed 202. In other aspects, the main reservoir 116 may also include an inner EDTA coating 114A as illustrated in FIG. 1.

In various aspects, the protective coatings described above provide the surgical drain system with the ability to collect drainage fluid samples containing intact analytes that may be analyzed in accordance with a variety of methods described in additional detail below. In some aspects, the drainage fluid may be analyzed to detect non-cellular RNA or DNA within a drainage fluid sample. Without being limited to any particular theory, the amounts of non-cellular RNA or DNA are typically present at low concentrations within a drainage fluid sample, and consequently the inhibition of DNA/RNA denaturing enhances the sensitivity of the methods of detection.

In various aspects, the surgical drain system may be further provided with additional features that facilitate the collection of high-quality surgical drainage fluid samples that preserve without degradation various components of the surgical drain fluid for analysis as described below. Non-limiting examples of suitable components that may be collected using the surgical drain system include circulating tumor cells, exosomes, and cell-free DNA (cfDNA) and RNA. In various aspects, the cell-free DNA and RNA may be associated with a variety of sources including, but not limited to, tumors, solid transplant organs, bacteria, fungi, and viruses within the surgical bed and/or within close proximity to the surgical bed.

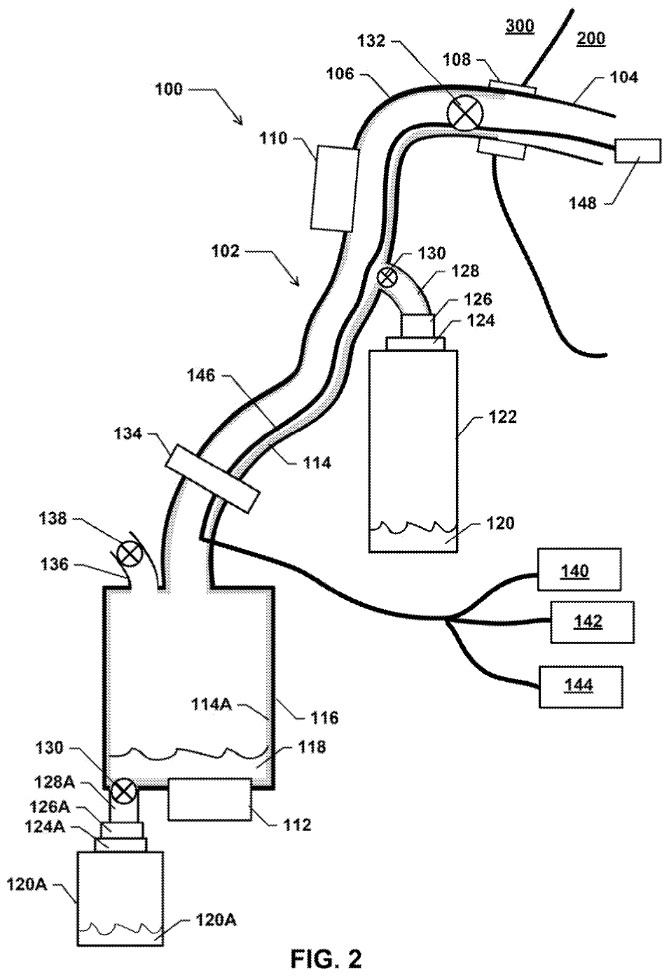

FIG. 2 is a schematic illustration of a surgical drain system 100A that is provided with one or more sample ports 128/128A to collect drainage fluid samples 120/120A within accessory reservoirs 122/122A. In some aspects, the accessory reservoirs 122/122A are configured to attach and detach from the corresponding sample ports 128/128A to provide for removal and replacement of the accessory reservoirs 122/122A. By way of non-limiting example, the accessory reservoir 122 may be removed from the sample port 128 when a sufficient amount of drainage fluid sample 120 has been collected and replaced with an empty replacement accessory reservoir (not illustrated). After removal, the accessory reservoir 122 may be subjected to an analysis including, but not limited to, NGS. In some aspects, the accessory reservoir 122 may be provided with additional features to facilitate the introduction of the drainage fluid sample 120 to the equipment used to perform the analysis of the sample 120.

Referring again to FIG. 2, the surgical drain system 100A may include a sample port 128 operatively coupled to the distal portion 106 of the drainage tube 102 in some aspects. In other aspects, the surgical drain system 100A may include a sample port 128A operatively coupled to the main reservoir 116. The sample ports 128/128A are configured to divert a portion of the drainage fluid 118 to form the drainage fluid samples 120/120A. As illustrated in FIG. 2, the sample ports 128/128A are provided with valves 130/130A that may be opened to divert the drainage fluid 116 to form the samples 120/120A. The valves 130/130A may also be closed to seal the sample ports 128/128A during removal and/or replacement of the accessory reservoirs 122/122A. In various other aspects, the sample ports 128/128A further include reversibly sealable connection fittings 126/126A configured to interlock and seal with corresponding features formed in the accessory reservoirs 122/122A to form sealed and reversible connections between the sample ports 128/128A and the accessory reservoirs 122/122A. Any suitable medical-grade connection fitting may be selected for use as reversibly sealable connection fittings including, but not limited to, Luer connectors.

In some aspects, the accessory reservoirs may be provided with additional features to enhance the quality of the drainage fluid samples and associated analysis methods. Referring again to FIG. 2, the accessory reservoirs 122/122A may further include sample filters 124/124A operatively coupled to the inlets of the accessory reservoirs 122/122A. The sample filters 124/124A are configured to remove one or more components from the drain fluid 118 including, but not limited to, whole cells, clots, and any other component that may potentially interfere with the operation of the assay devices and/or subsequent processing and analysis of the drain fluid samples 120/120A as described herein. The sample filters may facilitate the collection, centrifuging, and removal of necrotic debris from the drainage fluid samples 120/120A.

Any suitable medical-grade filter may be selected for use in the surgical drain system without limitation. In various additional aspects, the inner surfaces of the sample ports 128/128A and accessory reservoirs 122/122A may be coated with a coating composition (not illustrated) to inhibit denaturing or coagulation of analytes within the drain fluid samples 120/120A. Non-limiting examples of suitable coating compositions include EDTA, heparin, trisodium citrate (TSC) and any other suitable coating compound, and any combination thereof. In other aspects, the accessory reservoirs 122/122A and other elements of the surgical drainage system 100/100A may further contain one or more agents designed to preserve tumor-associated exosomes within the surgical drain tube or reservoirs.

In other aspects, the surgical drain system may further include features to maintain the drainage fluid at a suitable temperature within the drainage tube 102. Without being limited to any particular theory, DNA and RNA within the drainage fluid may degrade if the drainage fluid falls outside a suitable temperature range. Referring again to FIG. 2, the surgical drain system 100A may further include a heat source 140 configured to maintain the distal portion 106 of the drainage tube 102 within a temperature range suitable for maintaining the integrity of DNA and RNA molecules within the surgical drainage 118. The surgical drain system 100A may further include a thermal insulation material (not illustrated) positioned around at least a portion of the distal portion 106 of the drainage tube 102 to facilitate the maintenance of the drainage tube 102 within the suitable temperature range.

Any suitable heating device may be selected for use as a heat source 140 without limitation. Non-limiting examples of suitable heating devices include resistive heaters, piezoelectric heaters, water jacket heaters, and any other suitable heating device.

In other additional aspects, the surgical drain system may further include features to monitor various physiological parameters within the surgical bed indicative of a condition within the surgical bed. The surgical drain system may provide for monitoring of any suitable physiological parameter within the surgical bed without limitation including, but not limited to, temperature, pH, pressure, hydration, oxygenation, any other suitable physiological parameter, and any combination thereof.

Referring again to FIG. 2, the surgical drain system 100A may further include at least one physiological sensor 148 positioned at the proximal end 104 of the drainage tube 102. The at least one physiological sensor 148 is configured to monitor at least one physiological parameter within the surgical bed 202 as described above. In various aspects, the at least one physiological sensor 148 may be provided as a single sensor incorporating at least one probes to monitor the physiological parameter, or the at least one physiological sensor may be provided in the form of a separate probe for monitoring each physiological parameter within the surgical bed. In some aspects, the at least one physiological sensor may be operatively coupled to at least one physiological sensing device including, but not limited to, a pH monitor 142 and a temperature monitor 144.

In various aspects, the at least one physiological sensing device may be connected to the at least one physiological sensor 148 within the surgical bed 202 using any known communication device or protocol without limitation. Non-limiting suitable communication devices or protocols include connecting cables, wireless communication such as Bluetooth communication protocol, and any other suitable communication device or protocol. Referring again to FIG. 2, the at least one physiological probe 148 may be operatively coupled to the at least one physiological monitor device 142/144 using a cable 146 passed through the drainage tube 102.

In various additional aspects, methods for obtaining sequencing or molecular measures indicative of a condition of a surgical bed based on analysis of surgical drainage sample are disclosed. The methods include obtaining the surgical drainage sample using the surgical drain system disclosed herein. The disclosed methods further include analyzing the surgical drainage sample using at least one analysis method. Non-limiting examples of suitable methods of analysis include whole-genome sequencing, targeted sequence capture, multiplex PCR, methylation & 16S droplet PCR, and exosomal analysis. The disclosed methods obtain at least one sequencing or molecular measure of the condition of the surgical bed including, not limited to, residual cancer, infection, immune environment, transplant rejection, and risk of poor wound healing.

By way of non-limiting example, the surgical drainage system disclosed herein may be used, in combination with the analysis methods described above, for the collection, preservation, and quantification of tumor-associated cells or nucleic acids/cfDNA as measures of residual cancer, bacteria or virus-derived nucleic acids as early measures of wound infection, and transplant-derived nucleic acids as measures of transplant rejection. By way of an additional non-limiting example, the surgical drainage system disclosed herein may be used, in combination with the analysis methods described above, may be used for the collection and preservation of immune cells or immune-derived nucleic acids as measures of systemic or tumor immunity.

In various aspects, the disclosed surgical drainage system is suitable for use as a surgical drain for a variety of surgical sites and surgery types including, but not limited to, breast cancer resection and lymph node dissection, neck dissection, thoracic surgery for lung cancer with chest tube drain placement, abdominal surgery, colorectal cancer resection, pancreatic cancer resection (Whipple procedure), gynecologic cancer surgery, or prostate surgery requiring drain placement.

In various aspects, the surgical drainage system may be used in place of any suction-based surgical drainage device without limitation. In some aspects, the surgical drainage system may be adapted for use as a wound vac device configured for insertion into wounds.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Any publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

What is claimed is:

1. A surgical drainage system, comprising a device operatively coupled to a surgical drainage tube, the drainage tube configured for implantation within a surgical bed of a subject to drain surgical drain fluid out of the surgical bed, the device configured to detect at least one analyte within the drainage fluid, wherein the analyte is indicative of a condition.

2. The system of claim 1, wherein the surgical drain tube comprises a suction drain tube.

3. The system of any preceding claim, wherein the assay device comprises a lateral flow antigen test.

4. The system of any preceding claim, wherein the assay device further comprises a replaceable cartridge housing the lateral flow antigen test.

5. The system of any preceding claim, wherein the at least one analyte detected by the assay device comprises at least one biomarker indicative of the presence of at least one of bacteria, chyle, blood, bile, and any combination thereof.

6. The system of any preceding claim, wherein the at least one biomarker is selected from lipase, protease, peptidase, amylase, pepsin, trypsin, chymotrypsin, carboxypeptidase, elastase, phospholipase, lactase, maltase, sucrase, triglyceride, chylomicron, bilirubin, and any combination thereof.

7. The system of any preceding claim, wherein detection of the at least one analyte by the assay device indicates a surgical bed condition selected from an infection within the surgical bed, a formation of a fistula, a chyle leak, a bile leak, an anastomotic leak, and any combination thereof.

8. A surgical drainage system, comprising a surgical drainage tube comprising a proximal portion configured for implantation within a surgical bed of a subject and a distal portion to drain surgical drain fluid out of the surgical bed through an inner lumen defined within the proximal and distal portions of the surgical drainage tube, wherein at least a portion of the lumen is coated with a nucleic acid preservation agent.

9. The system of claim 8, further comprising a one-way valve positioned within the lumen, the one-way valve configured to prevent a backflow of surgical drain fluid within the lumen toward the surgical bed.

10. The system of any one of claims 8-9, wherein the surgical drainage tube is a suction surgical drainage tube.

11. The system of any one of claims 8-10, wherein the nucleic acid preservation agent is selected from ethylene diamine tetra acetic acid (EDTA), sodium dodecyl sulfate (SDS), and any combination thereof.

12. The system of any one of claims 8-11, further comprising a sample port operative connected to the lumen of the surgical drainage tube, the sample port comprising a valve and a connection fitting, the sample port configured seal when the valve is in a closed position and to direct a sample out of the lumen when the valve is in an open position, the sample comprising a portion of the surgical drain fluid.

13. The system of any one of claims 8-12, further comprising an accessory reservoir reversibly coupled to connection fitting, wherein the accessory reservoir is configured to receive the sample from the sample port.

14. The system of any one of claims 8-13, wherein the accessory reservoir is lined with the nucleic acid preservation agent.

15. The system of any one of claims 8-14, wherein the connection fitting comprises a resealable lock configured to reversibly couple the accessory reservoir to the connection fitting.

16. The system of any one of claims 8-15, wherein the resealable lock comprises a Luer lock.

17. The system of any one of claims 8-16, wherein sample port is further configured to reversibly couple and detach from the accessory reservoir and at least one replacement accessory reservoir for serial sampling.

18. The system of any one of claims 8-17, wherein the accessory reservoir further comprises a sample filter configured to filter the surgical drain fluid passing into the accessory reservoir.

19. The system of any one of claims 8-18, further comprising a heat source operatively coupled to at least a portion of the surgical drainage tube and to at least a portion of the accessory reservoir, the heat source configured to maintain the surgical drain fluid within the lumen and the surgical drain fluid sample within the accessory reservoir at a temperature selected to maintain integrity of nucleic acids within the surgical drain fluid and surgical drain fluid sample.

20. The system of any one of claims 8-19, further comprising a thermal insulation material enclosing at least a portion of the surgical drainage tube and the accessory reservoir.

21. The system of any one of claims 8-20, further comprising at least one physiological sensor positioned at the proximal end of the surgical drain tube, each physiological sensor configured to monitor a physiological condition of the surgical bed.

22. The system of any one of claims 8-21, wherein the at least one physiological sensor monitors a physiological condition independently selected from temperature, pH, pressure, hydration, oxygenation, and any combination thereof.

23. The system of any one of claims 8-22, further comprising an assay device operatively coupled to the lumen within the distal portion of the surgical drainage tube, the assay device configured to detect at least one analyte within the drainage fluid, wherein the at least one analyte is indicative of a surgical bed condition.

24. The system of any one of claims 8-23, wherein the assay device comprises a lateral flow antigen test.

25. The system of any one of claims 8-24, wherein the assay device further comprises a replaceable cartridge housing the lateral flow antigen test.

26. The system of any one of claims 8-25, wherein the at least one analyte detected by the assay device comprises at least one biomarker indicative of the presence of at least one of bacteria, chyle, blood, bile, and any combination thereof.

27. The system of any one of claims 8-26, wherein the at least one biomarker is selected from lipase, protease, peptidase, amylase, pepsin, trypsin, chymotrypsin, carboxypeptidase, elastase, phospholipase, lactase, maltase, sucrase, triglyceride, chylomicron, bilirubin, and any combination thereof.

28. The system of any one of claims 8-27, wherein detection of the at least one analyte by the assay device indicates a surgical bed condition selected from an infection within the surgical bed, a formation of a fistula, a chyle leak, a bile leak, an anastomotic leak, and any combination thereof.
